(19) United States
(12) Patent Application Publication
(10) Pub. No.: US 2009/0162406 A1
(43) Pub. Date: Jun. 25, 2009

(54) WOUND HEALING WITH ZEOLITE-BASED HEMOSTATIC DEVICES

(75) Inventors: Giacomo Basadonna, Haddam, CT (US); Raymond J. Huey, Orange, CT (US); Denny Lo, Bethlehem, CT (US)

Correspondence Address: KNOBBE MARTENS OLSON & BEAR LLP, 2040 MAIN STREET, FOURTEENTH FLOOR, IRVINE, CA, 92614, US

(73) Assignee: Z-MEDICA CORPORATION, Wallingford, CT (US)

(21) Appl. No.: 12/204,129

(22) Filed: Sep. 4, 2008

Related U.S. Application Data
(60) Provisional application No. 60/967,510, filed on Sep. 5, 2007.

(51) Int. Cl. A61K 9/00; A61K 33/06; A61K 33/00; A61P 17/02

(52) U.S. Cl. 424/400; 424/684; 424/724

(57) ABSTRACT

A method for decreasing the time it takes for a wound to heal includes applying hemostatic agent to the wound, inflaming tissue surrounding the wound to facilitate the deposition of fibroblast, thereby accelerating the subsequent contraction of the wound and the onset of the proliferative healing stage, and causing the re-epithelization of the tissue at a faster rate than if no hemostatic agent was applied. A method for promoting the healing of a bleeding wound includes coating a hemostatic agent onto a substrate, applying the substrate to the bleeding wound so that an effective amount of the hemostatic agent is applied to the wound, inflaming the tissue, and causing the re-epithelization of the tissue at a faster rate than if no hemostatic agent was applied. In at least some methods, a clotting cascade and platelet aggregation within the bleeding wound is accelerated, and blood loss from the wound is decreased.

### CROSS REFERENCE TO RELATED APPLICATION

The present application claims the benefit of U.S. Provisional Patent Application No. 60/967,510, filed Sep. 5, 2007, the contents of which are incorporated herein by reference in their entirety.

### TECHNICAL FIELD

The present invention relates generally to wound healing and, more particularly, to methods of promoting wound healing using zeolite-based hemostatic devices.

### BACKGROUND OF THE INVENTION

Blood is a liquid tissue that includes red cells, white cells, corpuscles, and platelets dispersed in a liquid phase. The liquid phase is plasma, which includes acids, lipids, solublized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of circumstances, animals, including humans, can be wounded. Often bleeding is associated with such wounds. In some circumstances, the wound and the bleeding are minor, and normal blood clotting functions in addition to the application of simple first aid are all that is required. Unfortunately, however, in other circumstances substantial bleeding can occur. These situations usually require specialized equipment and materials as well as personnel trained to administer appropriate aid. If such aid is not readily available, excessive blood loss can occur. When bleeding is severe, sometimes the immediate availability of equipment and trained personnel is still insufficient to stop the flow of blood in a timely manner.

Once the bleeding is stopped, the process of wound healing can begin. This process is the body's primary mechanism for repairing dermal or epidermal damage. The process is categorized into three steps, namely, (1) the inflammatory phase; (2) the proliferative phase; and (3) the remodeling phase. These steps are largely sequential, but they can overlap in time to some degree. The inflammatory phase typically ranges from the immediate infliction of the wound to 2-5 days; the proliferative phase typically ranges from about 2 days to about 3 weeks; and the remodeling phase typically ranges from about 3 weeks to about 2 years. Events in the inflammatory phase include hemostasis; phagocytosis of bacteria, debris, and damaged tissue; and release of blood clotting factors (e.g., Factor VIII, Factor IX, and Factor XI) that cause platelets to aggregate, thereby inducing the proliferative stage. Events in the proliferative phase include the growth of new blood vessels, collagen deposition, new tissue formation, and wound contraction. In the remodeling phase, epithelial cells grow across the wound to form a covering.

When left to heal on their own, wounds tend to proceed through these three steps at a leisurely pace and according to several factors. These factors include the specific makeup of the blood, age of the wounded person, and specific details relating to the wounded tissue such as hydration levels, location of the wound, manner of acquiring the wound, the nutritional intake of the wounded person, etc. Oftentimes, allowing a wound to heal without intervention to facilitate and speed up the healing process can result in infection setting in increased pain and discomfort to the wounded person, and/or prolonged undesirable drug therapy.

Based on the foregoing, what is needed is a method of speeding up the healing process to limit the undesirable effects thereof.

### SUMMARY OF THE INVENTION

In one aspect, the present invention is directed to a method for decreasing the time it takes for a wound to heal. In this method, an effective amount of a hemostatic agent is applied to the wound. In doing so, the inflammation of the tissue surrounding the wound is increased to facilitate the deposition of fibroblast, thereby accelerating the subsequent contraction of the wound and the onset of the proliferative healing stage. The re-epithelization of the tissue can then occur at a faster rate than if no hemostatic agent was applied to the wound.

In another aspect, the present invention is directed to a method for promoting the healing of a bleeding wound. In this method, a hemostatic agent is coated onto a substrate, which is in turn applied to the bleeding wound so that an effective amount of the hemostatic agent is applied to the wound. In doing so, the tissue at, in, around, or in proximity to the wound is inflamed, and fibroblast is deposited to the tissue of the wound. The tissue is then re-epithelized at a faster rate than if no hemostatic agent was applied.

In another aspect, the present invention is directed to a method of accelerating the healing of a bleeding wound. In this method, a hemostatic agent is applied to a bleeding wound to facilitate a healing process. In doing so, a clotting cascade and platelet aggregation within the bleeding wound is accelerated, and blood loss from the wound is decreased, thereby causing local inflammation of tissue at the bleeding wound and the subsequent contraction of the tissue. The inflammation and contraction causes an increase in fibroblast deposition. Utilization of this process provides for an increase in the time it takes to heal the bleeding as compared to a bleeding wound in which a hemostatic agent is not applied.

One advantage of the present invention is that the wound heals more quickly than a wound that is not treated with hemostatic agent. In particular, the increase in fibroblast deposition at the wound site accelerates the healing process.

Another advantage of the present invention is that the risks associated with wound healing, namely, the onset of infections that delay the healing process, are reduced. Because the blood emanating from the wound clots more quickly than if no hemostatic agent was used, a coagulum plug forms over the wound more quickly to form a bacteria resistant barrier.

### BRIEF DESCRIPTION OF THE DRAWINGS

FIG. 1 is a cross-sectional view of a particle of hemostatic agent; and

FIG. 2 is a graphical representation showing re-epithelization rates for wounds left untreated and wounds treated with hemostatic agent.

### DETAILED DESCRIPTION OF THE PREFERRED EMBODIMENTS

Disclosed herein are methods for decreasing the healing time of a wound following acute or chronic injuries by improving conditions for the initial stages of wound healing and accelerating the subsequent phases of wound healing. The methods typically employ compositions that are applied in effective quantities to bleeding wounds to promote hemostasis. These compositions generally comprise hemostatic agents as active ingredients that can minimize or stop blood flow by absorbing at least portions of the liquid phases of the blood, thereby promoting clotting.

In one embodiment of the present invention, the hemostatic agent is a zeolite or other molecular sieve material. The present invention is not limited in this regard, however, as other materials are also within the scope of the present invention. As used herein, the term “zeolite” refers to a crystalline form of aluminosilicate having the ability to be dehydrated without experiencing significant changes in the crystalline structure. The zeolite may include one or more ionic species such as, for example, calcium and sodium moieties. The preferred molecular structure of the zeolite is an “A-type” crystal, namely, one having a cubic crystalline structure that defines round or substantially round openings.

The zeolite may be mixed with or otherwise used in conjunction with other materials having the ability to be dehydrated without significant changes in crystalline structure. Such materials include, but are not limited to, magnesium sulfate, sodium metaphosphate, calcium chloride, dextrin, combinations of the foregoing materials, and hydrates of the foregoing materials.

Zeolites for use in the disclosed applications may be naturally occurring or synthetically produced. Numerous varieties of naturally occurring zeolites are found as deposits in sedimentary environments as well as in other places. Naturally occurring zeolites that may be applicable to the compositions described herein include, but are not limited to, analcite, chabazite, heulandite, natrolite, stilbite, and thomosonite. Synthetically produced zeolites that may also find use in the compositions and methods described herein are generally produced by processes in which rare earth oxides are substituted by silicates, alumina, or alumina in combination with alkali or alkaline earth metal oxides.

Various materials may be applied to the wound in conjunction with the zeolite or other hemostatic agent by being mixed with, associated with, or incorporated into the zeolites to maintain an antiseptic environment at the wound site or to provide functions that are supplemental to the clotting functions of the zeolites. Exemplary materials that can be used include, but are not limited to, pharmaceutically-active compositions such as antibiotics, antifungal agents, antibacterial agents, antimicrobial agents, anti-inflammatory agents, analgesics, antihistamines (e.g., cimetidine, chloropheniramine maleate, diphenhydramine hydrochloride, and promethazine hydrochloride), iodine, compounds containing silver ions, and the like. The antibacterial ingredients in particular promote the healing process by decreasing the proliferation of bacteria in the wound. Other materials that can be incorporated to provide additional hemostatic functions include ascorbic acid, tranexamic acid, rutin, and thrombin. Botanical agents having desirable effects on the wound site may also be added.

The zeolite or hemostatic agent may be applied to an inert substrate or vehicle to be applied to a bleeding wound. For application to such an inert substrate or vehicle, the zeolite or other hemostatic agent is preferably in powder form. The powder form of the zeolite may be obtained by any suitable operation. For example, powdered zeolite may be obtained by grinding, crushing, rolling, or pulverizing coarser zeolite material. The present invention is not limited in this regard, however, as other methods of manipulating the zeolite into powder form known to those skilled in the art in which the present invention pertains may be employed.

In another embodiment of the present invention, the hemostatic agent coated onto the substrate is a bioactive glass. As used herein, the term “bioactive glass” refers to a surface-reactive glassy ceramic material that is biocompatible with human tissue. The composition of bioactive glass promotes a rapid ion exchange in aqueous environments. Bioactive glass can be defined by any one of a multitude of formulas, but it is predominantly a mixture of oxides. In general, bioactive glasses include silicon dioxide and calcium oxide. Other materials that may be incorporated into the bioactive glasses include, but are not limited to, sodium oxide and phosphorous pentoxide. Still other materials that may be added to the bioactive glasses include, but are not limited to, the pharmaceutically-active compositions described above.

In other embodiments, the material coated onto the substrate may be a siliceous oxide, a mixture of various siliceous oxides, any type of mesoporous material, a clay (e.g., attapulgite, bentonite, kaolin, or combinations thereof), diatomaceous earth, a biological composition having hemostatic characteristics (e.g., chitosan, thrombin, fibrin, Factor VII or similar enzymes, or compositions thereof), or any other composition having hemostatic properties. Such materials may be used in combination with zeolites or other molecular sieve materials.

Although the compositions and their methods of manufacture are described herein with reference to the active ingredient being a zeolite, it should be understood by those of skill in the art that the hemostatic agents and their methods of manufacture may additionally incorporate a bioactive glass, a siliceous oxide, a mesoporous material, a clay, diatomaceous earth, biological compositions, or any combination thereof to define the active ingredient.

In formulating the hemostatic agent, the zeolite is adhered to the substrate. The mechanism for adhesion between the zeolite and the substrate materials may be coulombic forces, a separate binding material, or an additional hemostatic agent. In embodiments in which a separate binding material is used, the material may be any biocompatible composition having sufficient properties that allow the composition to be retained on the substrate and to retain the active ingredient.

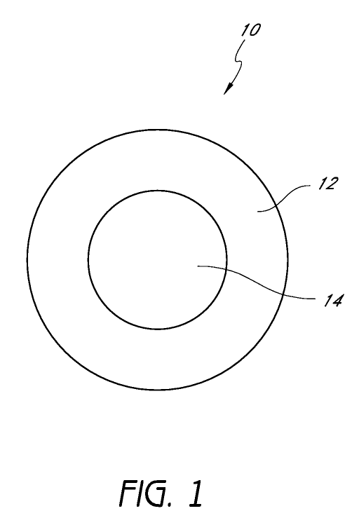

Referring now to FIG. 1, a hemostatic agent is shown generally at 10. In one exemplary embodiment, the hemostatic agent 10 comprises the zeolite, shown at 12, disposed on the substrate 14. The substrate 14 may be clay, an artificial or processed gel or gelling agent, or some other type of material such as a plastic that binds the zeolite 12 thereto or otherwise holds the zeolite. An additional binder may also be used to adhere the zeolite 12 to the substrate 14.

Zeolite-based hemostatic agents facilitate hemostasis, which in turn accelerates the proceeding of the clotting cascade and platelet aggregation. Such agents also promote wound healing following acute and chronic (including ischemic) injuries by improving the inflammatory stage of wound healing to more rapidly allow the beginning of the proliferative phase. The agents therefore decrease blood loss and the associated risk of complications such as infections that might delay wound healing. In addition, the agents cause local inflammation which increases fibroblast deposition and wound contraction.

Another application of the agents in the healing of wounds involves debridement, which is the surgical or mechanical removal of infected tissue from a wound. This procedure is sometimes used on chronic wounds to promote the healing of the healthy tissue, but is known to cause significant bleeding as a result of tissue removal. The agents serve to stop the bleeding and kill bacteria by direct contact (if an antibacterial version of the device is used).

### Example

### Comparison of Wound Healing Rates

In one study, a zeolite-based hemostatic agent was used to treat deep partial thickness wounds created on porcine subjects. The hemostatic agent was disposed in a pouch that allowed for the flow of blood therethrough to contact the hemostatic agent. The pouch was applied to each porcine subject in one of three manners. First, the pouch was applied to a wound for three minutes, then the wound was covered with gauze. Second, the pouch was applied to a wound for 24 hours each day and covered with gauze. In this second manner, the hemostatic agent and the pouch were changed after each 24 hour period. Third, a wound was made in the porcine subject and not treated. After four days, the wounds were evaluated for epithelization and compared. The wounds treated with zeolite-based hemostatic agent had higher rates of epithelization than the untreated wounds.

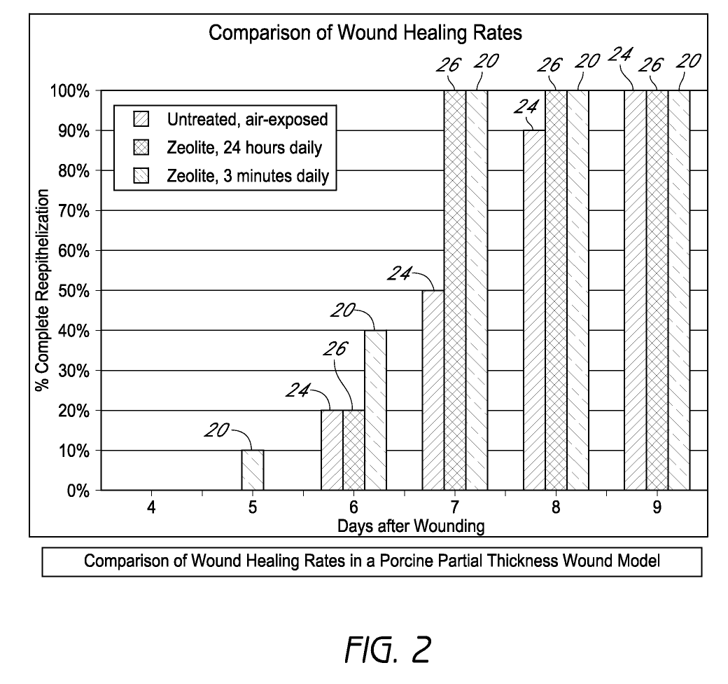

Referring to FIG. 2, in a comparison of wound healing rates, it can be seen that the re-epithelization process proceeded at a faster rate when the hemostatic agent was applied for three minutes daily than it did when the hemostatic agent was applied for a 24 hour period, which was by comparison faster than when there was no treatment applied. In particular, in instances in which the hemostatic agent was applied for three minutes daily, as shown by the bar graph 20, re-epithelization (about 10%) was noted after about five days, whereas in the untreated wound (bar graph 24) and in the wound in which hemostatic agent was applied for 24 hour periods (bar graph 26), re-epithelization was first noted after about six days. Complete re-epithelization was noted for both the hemostatic agent-treated wounds after about seven days, whereas at about 7 days untreated wounds still only experienced about 50% re-epithelization.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A method for decreasing the healing time of a wound, said method comprising the step of:
applying an effective amount of a hemostatic agent to said wound to increase the inflammation of tissue surrounding said wound, thereby causing the deposition of fibroblast and the acceleration of a re-epithelization of said tissue of said wound.

2. The method of claim 1, wherein said hemostatic agent is a molecular sieve material.

3. The method of claim 2, wherein said molecular sieve material is a zeolite.

4. The method of claim 1, wherein said hemostatic agent is a bioactive glass.

5. The method of claim 1, further comprising applying a pharmaceutically-active composition to said wound in conjunction with said hemostatic agent.

6. The method of claim 1, further comprising the step of dehydrating said hemostatic agent prior to application to said wound.

7. The method of claim 1, wherein said hemostatic agent is a bioactive glass.

8. The method of claim 1, further comprising the step of applying said hemostatic agent to an inert substrate.

9. The method of claim 8, wherein said step of applying the effective amount of the hemostatic agent to said wound comprises applying said inert substrate to said wound.

10. A method for promoting the healing of a bleeding wound, said method comprising the steps of:
providing a substrate on which a hemostatic agent is coated;
applying said substrate to a bleeding wound such that an effective amount of said hemostatic agent is applied to said bleeding wound;
inflaming tissue proximate said bleeding wound, thereby accelerating a deposition of fibroblast and accelerating a subsequent contraction of said tissue; and
causing a re-epithelization of said tissue;
wherein said step of causing a re-epithelization of said tissue occurs at a faster rate than if no hemostatic agent was applied to said bleeding wound.

11. The method of claim 10, wherein said hemostatic agent is selected from the group consisting of molecular sieve material, zeolite, bioactive glass, siliceous oxide, mixtures of siliceous oxides, mesoporous material, clay, diatomaceous earth, chitosan, and combinations of the foregoing materials.

12. The method of claim 10, wherein said substrate is selected from the group consisting of clay, gel, gelling agent, and plastic.

13. The method of claim 10, further comprising the step of maintaining said substrate in contact with said bleeding wound for a pre-selected period of time.

14. The method of claim 10, further comprising the step of debriding said bleeding wound.

15. A method of accelerating the healing of a bleeding wound, said method comprising the steps of:
applying a hemostatic agent to a bleeding wound to facilitate a healing process;
accelerating a clotting cascade and platelet aggregation within said bleeding wound;
decreasing blood loss from said bleeding wound;
causing local inflammation of tissue at said bleeding wound to increase fibroblast deposition; and
causing a contraction of said tissue at said bleeding wound;
wherein an increase in healing time of said bleeding wound is facilitated as compared to a bleeding wound in which a hemostatic agent is not applied.

16. The method of claim 15, further comprising debriding said tissue of said bleeding wound.

17. The method of claim 15, wherein said wound is healed via a re-epithelization of said tissue of said bleeding wound.

18. The method of claim 15, wherein said step of applying said hemostatic agent comprises a step of applying a zeolite to said bleeding wound.

19. The method of claim 15, wherein said step of applying said hemostatic agent comprises a step of applying a bioactive glass to said bleeding wound.

20. The method of claim 15, wherein said step of applying said hemostatic agent comprises a step of applying said hemostatic agent on an inert substrate.
